(19) United States
(12) Patent Application Publication
(10) Pub. No.: US 2016/0015320 A1
(43) Pub. Date: Jan. 21, 2016

(54) Smart Medical Device for Electrochemical Monitoring and Control of Medical Implants

(71) Applicant: Jeremy GILBERT, Fayetteville, NY (US)

(72) Inventor: Jeremy Gilbert, Fayetteville, NY (US)

(21) Appl. No.: 14/774,761

(22) PCT Filed: Mar. 17, 2014
(86) PCT No.: PCT/US2014/030271

§ 371 Date: Sep. 11, 2015

Related U.S. Application Data
(60) Provisional application No. 61/794,595, filed on Mar. 15, 2013.

(51) Int. Cl. A61B 5/00; A61N 1/36; A61B 5/053

(52) U.S. Cl. CPC A61B 5/4851; A61B 5/053; A61N 1/36014

(57) ABSTRACT

An external or implantable system for measuring for measuring electrical factors, such as voltage, current, and impedance, to assess the behavior of metallic biomaterials surfaces and to determine the corrosion-based activity of the surfaces while placed in their normal location within the human body. The system includes electrodes for interrogating the medical implant and electronics for monitoring the implant and controlling the electrodes, as well as a power source and communication module.

### CROSS-REFERENCE TO RELATED APPLICATIONS

This application claims priority to U.S. Provisional Application No. 61/794,595, filed on Mar. 15, 2013.

### BACKGROUND OF THE INVENTION

1. Field of the Invention

The present invention relates to implantable medical devices and, more specifically, to a system and method for monitoring corrosion of implanted medical devices using electrochemical changes.

2. Description of the Related Art

Medical implants, such as metallic biomaterials implanted in the body, can experience a range of stimuli that can alter the magnitude and nature of electrochemical reactions that take place at the implant surface. For example, when a metallic biomaterial has its surface abraded by an opposing hard surface, e.g., a wear process is taking place, the corrosion reactions can take place at the surface of the implant and dramatically alter the structure of the device. The conventional approach for evaluating metallic medical devices is to use the existing clinical diagnostic tools for assessing implant corrosion status. For example, x-rays, verbal inquiries of the patient, and, perhaps even blood and urine testing for metal ion levels may be used. These approaches are generally insufficient for determining the severity of wear and/or corrosion processes taking place at implant surfaces.

In terms of control of corrosion and infections, there are no available approaches for controlling implant-centered corrosion and infections. Instead, current methods require implant removal, extensive debridement of the infected site, introduction of an antibiotic releasing temporary component, and after several months subsequent reoperation after the infraction has resolved.

### BRIEF SUMMARY OF THE INVENTION

The present invention comprises a sensor system for taking electrochemical measurements (e.g., micro-impedance measurements) of an implanted medical device to perform a direct measurement of the voltage, current and/or impedance of regions of corroded modular taper interfaces to assess severity of corrosion damage or infection. The present invention includes electrodes for measurement, monitoring, electronic circuitry to assess voltage, current and impedance and systems for acquisition, storage and transmission of electrochemical data. Electrochemical factors, such as voltage, current, and impedance, can be measured to assess the behavior of metallic biomaterials surfaces and to determine the corrosion-based activity of the surfaces while placed in their normal location within the human body. The present invention may be implemented in a testing module to be used at the time that an implanted medical device is exposed during a surgical revision procedure, or miniaturized and embedded in the medical device itself prior to implantation.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of a system for taking micro-impedance measurements of an implanted medical device according to the present invention;

FIG. 2 is a schematic of an embodiment of the present invention incorporated into an implanted medical device.

### DETAILED DESCRIPTION OF THE INVENTION

Referring now to the drawings, wherein like reference numerals refer to like parts throughout, the present invention comprises a system 10 having electrodes for measuring electrochemical factors, such as voltage, current, and impedance, to assess the behavior of metallic biomaterials surfaces and to determine the corrosion-based activity of the surfaces while placed in their normal location within the human body. Electrodes may include reference electrodes and counter electrodes to complete the electrochemical circuitry in the body.

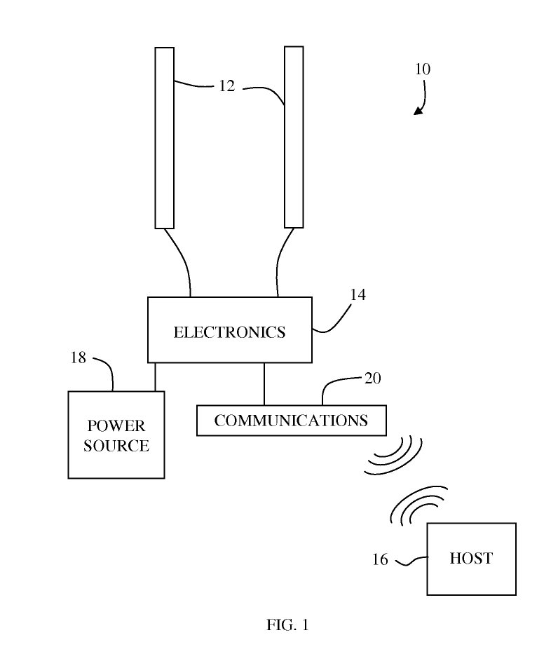

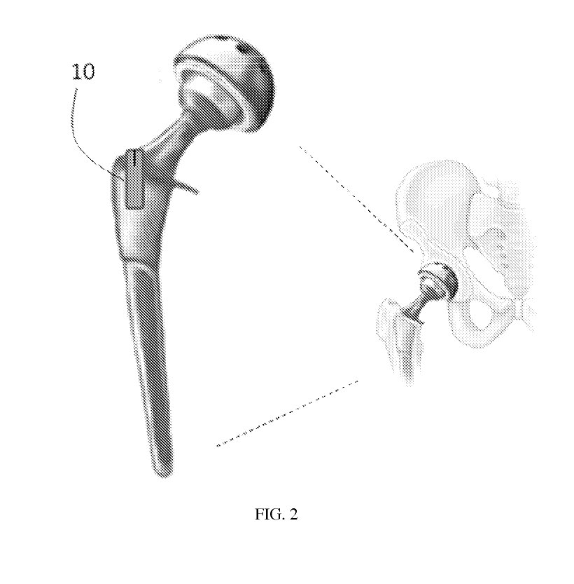

System 10 may be provided an external testing assembly used by a surgical team after a medical implant has been exposed, or an internal module provided as part of the medical implant itself. Referring to FIG. 1, system 10 includes electrodes 12 that can be used to interrogate the implant (e.g., reference and counter electrodes), and electronics 14 for the control, monitoring, power, and communication elements of system 10. Electronics 14 can include microelectronic integrated circuits designed specifically for electrochemical control and monitoring. System 10 also required a power source 18, which may be a radiofrequency induction coil, as well as a communication package 20, such as an RFID, NFC, or Bluetooth protocol communication module for digital communication between system 10 and an external host 16. It should be recognized that the various module could be implemented in independent circuits, a programmable microcontroller, programmed firmware, or a combination thereof. As seen in FIG. 2, system 10 may be integrated into a medical implant that is surgically implanted into a patient, e.g., a hip implant.

During the activities of daily living, metallic biomaterials implanted in the body can experience a range of stimuli that can alter the magnitude and nature of electrochemical reactions that take place at the implant surface. For example, when a metallic biomaterial has its surface abraded by an opposing hard surface, the corrosion reactions take place at the surface of the implant. As a result, currents arise and electrochemical voltages of the metal are altered in ways that are detected by the present invention. During abrasion, for example, the voltage of the surface, known as the open circuit potential, will become more negative (cathodic) as the surface oxide film is abraded and reformed by corrosion reactions. This shifting in voltage is a manifestation of the extent of ongoing corrosion damage occurring are a result mechanically assisted corrosion processes.

In the external embodiment of the present invention, the implant interrogation and control may be performed while the implant is exposed in an operating room during a revision surgery. System 10 may be used to assess the implanted device for damage by mechanically assisted corrosion processes by using electrochemical methods to determine the extent and severity of the damage present on the region of the implant that is most critical during revision, namely, the modular taper interfaces. These modular tapers are the location where replacement components are typically attached to those portions of the implant that will remain in place during revision surgery. The components that remain may be damaged, but are well integrated into the body and would cause severe harm if removed. Presently, the decision to remove or not these damaged components is left to the visual evaluation of the implant surface by the surgeon for signs of damage. The electrochemical measurements afforded by system 10 may thus be used to identify damage that is not clearly identifiable by the surgeon.

In the implantable embodiment of the present invention, system 10 generally comprises an on-board high impedance voltmeter circuit and an embedded reference electrode wire (e.g. a silver wire). The temporal variation of the voltage of this device can be tracked over time, stored in on-board storage medium, and ultimately relayed out through a transmission circuit to an external capture system. This will allow doctors, for example, to have patient implant performance monitored over a period of time and the information relayed out for interpretation of the severity of the corrosion reactions taking place in that implant.

Because of the major concerns about the corrosion of total joint replacements, resurfacing devices, and other metallic biomaterials, an implantable system 10 will provide the surgeon and the patient the ability to learn about the local environment and the electrochemical interaction of the device with that environment. Early detection of the onset of severe corrosion reactions can provide the surgeon with critical information in the diagnosis and treatment of conditions that are known to arise from the corrosion of metallic biomaterials including osteolysis and pseudotumors.

In addition to detecting and preventing the side effects of corrosion, there are other changes at the implant-body interface in metallic biomaterials that are amenable to interrogation with electrochemical systems. These include the detection of implant-centered infection as a bacterial biofilm that colonizes an implant surface may alter the impedance characteristics of the surface. Similarly, the loss of bony ingrowth may change the voltage or impedance behavior of the implant in a characteristic matter than can be detected and reported to the doctor and patient.

In addition to monitoring metallic biomaterials surfaces for electrochemical behavioral changes, system 10 may include on-board electronics adapted to impart electrochemical energy, i.e., voltages and currents, to elicit specific biological processes that are beneficial to the implant and the patient. For example, sufficient cathodic voltages applied to metallic biomaterial surfaces can result in controlled killing of bacterial and other cell types. Also, direct-current bone electrical stimulation can induce bone formation using cathodic electrochemical currents. The incorporating of these approaches into system 10 can allow a physician to induce specific electrochemical effects that benefit the patient, such as the killing of a nascent infection at the implant or stimulating bone formation at the implant-bone interface.

System 10 may thus be used for total joint replacements and other orthopedic, spinal, as well as dental applications and other systems requiting smart metallic biomaterials. System 10 addresses the problem of inadequate ability to clinically assess and diagnose corrosion-related issues with the use of metallic biomaterials, and provides a physician with quantitative data with which to assess the electrochemical status of an implant and to determine if unacceptable degradation rates are taking place long before other clinical diagnostic tools would be able to provide such information. System 10 also provides a new way to control and affect the processes and behaviors of the biological system adjacent to the implant by locally controlling the electrochemical environment and status of the implant. These effects may include speeding bone ingrowth, killing implant centered infections, altering inflammatory activity, and addressing other implant-related factors.

### EXAMPLE 1

System 10 may be designed and evaluated by preparing a self-containing envelope with electrodes for performing voltage and impedance evaluations on implanted modular taper junctions that are intended to remain in the patient without removal. The envelope may be made from a flexible polymer, such as silicone, which can be put over the male taper of a modular junction in a hip implant that has been damaged by fretting crevice corrosion processes. This would serve to electrically and electrochemically isolate the patient from the test. The electrochemical responses (voltage and impedance behavior) may then be evaluated over a range of damaged and corroded tapers and compared similar measurement from pristine surfaces. The differences in the responses from a range of damage levels in tapers will thus provide insight into sensitivity of the tests needed to be performed by system 10 to evaluate the extent of damage in a particular implant. Appropriate tests include a measurement of the impedance of the surface, which will vary with the level of corrosion damage. System 10 should preferably be connected to an external measurement host for tracking of measurements and reporting performance. Variations in electrode design (material, placement), isolation of device from patient environment, and most effective solution may also be explored in these tests. It will also be recognized by those of skill in the art that tests of system 10 may be needed to determine the effect of sterilization as system 10 may be used intraoperatively and thus would need to be sterilized and packaged appropriately.

### EXAMPLE 2

The implantable embodiment of system 10 will undoubtedly need extensive testing as the safety and efficacy of system 10 will need to be established. In addition, the appropriate design of electrical components and their packaging into system 10 will need to be evaluated. For example, if voltage measurements are the only signal to be acquired, then system 10 will require an onboard reference electrode and a high impedance voltage measurement circuit. System 10 would also need to be configured to be energized externally, such as with external radio frequency source, and configured to sample and report out the voltage between reference and implant. In this embodiment, an external radiofrequency source with a digital sample acquisition and storage system will be required, and thus tested in terms of performance and possible failure modes. Bench testing of system 10 should be the first step, followed by implantation into animals for sufficient periods of time to assess long term performance.

Animal studies would additionally allow for examination of the interaction of system 10 with a body environment, including wound healing effects (such as encapsulation). Specific tests of implants with known corrosion behavior would also be performed and monitored with system 10, and the associated tissue response could be directly compared to the electrochemical response.

### EXAMPLE 3

Animal tests could also be used to explore the active embodiment of system 10 where voltages can be applied and the response (current and voltage) can be collected and compared to biological response. For example, an implant surface could be pre-infected with a bacterial biofilm, and then sensor 10 used to apply voltages to the implant and to assess the extent of recovery from the infection at various times post-infection.

### EXAMPLE 4

A further evaluation of the active embodiment of system 10 could be performed by designing a fretting corrosion implant, i.e., a device that will purposefully engage in fretting corrosion reactions, and then implanting the fretting corrosion implant in an animal having sensor 10 also implanted. The electrochemical response to the corrosion processes may then be measured over time, with the local tissue response ultimately assessed. Different severities of fretting corrosion can be designed into the fretting corrosion implant, and system 10 tested for its ability to detect differences in corrosion.

### EXAMPLE 5

The present invention may be used to detect cell-based corrosion of cobalt-chromium-molybdenum (CoCrMo) alloy implants, which has recently been discovered as a possible cause of implant corrosion and the release of implant wear debris. A recently study of the corrosion morphology on CoCrMo implant surfaces and the presence of cellular remnants and biological materials intimately entwined with the corrosion, revealed that direct cellular attack may be the cause of the corrosion. In addition, the study found that a Fenton-like reaction mechanism may be responsible for corrosion in which reactive oxygen species are the major cause. For example, large increases in corrosion susceptibility of CoCrMo were seen (40 to 100 fold) when immersed in phosphate buffered saline solutions modified with hydrogen peroxide and HCl to represent the chemistry under inflammatory cells under the cell membrane region of adhered and/or migrating inflammatory cells. System 10 may thus be used to detect the presence of conditions that lead to inflammatory cell directed corrosion, such as the open circuit potential or electrochemical impedance spectroscopy of the implant.

What is claimed is:

1. A device for assessing the behavior of an implanted medical device, comprising:
a pair of electrodes configured to detect one or more electrochemical parameters of said implanted medical device and output at least one signal corresponding to said one or more electrical parameters;
a controller interconnected to said electrodes and programmed operate said electrodes and to receive said at least one signal from said electrodes;
a power source interconnected to said controller and said electrodes.

2. The device of claim 1, wherein said one or more electrical parameters comprises the open circuit potential of said implanted medical device.

3. The device of claim 1, wherein said controller comprises a high impedance voltmeter.

4. The device of claim 3, wherein said controller includes a storage medium.

5. The device of claim 4, wherein said controller is programmed to record the temporal variation of the voltage of the implanted medical device over time.

6. The device of claim 5, further comprising a transmission circuit interconnected to said microcontroller for wirelessly transmitting said temporal variation of the voltage of the implanted medical device to a remotely positioned host.

7. The device of claim 1, wherein said controller is programmed to apply electrochemical energy to said implanted medical device.

8. The device of claim 7, wherein said electrochemical energy applied to said implanted medical device comprises a cathodic voltage.

9. The device of claim 1, wherein said pair of electrodes, said controller, and said power source are positioned in an envelope and affixed to the implanted medical device.

10. A method of assessing the behavior of an implanted medical device, comprising the steps of:
positioning a device configured to measure one or more electrochemical parameters of said implanted medical device in close proximity to said implanted medical device;
measuring said one or more electrochemical parameters of said implanted medical device;
transmitting data representative of said one or more electrochemical parameters of said implanted medical device to a remotely positioned host.

11. The method of claim 10, wherein said one or more electrical parameters comprises the open circuit potential of said implanted medical device.

12. The method of claim 10, wherein said device comprises a high impedance voltmeter.

13. The method of claim 12, wherein said device further comprises a storage medium.

14. The method of claim 13, further comprising the step of recording the temporal variation of the voltage of the implanted medical device over time.

15. The method of claim 14, wherein said device further comprises a wireless transmission circuit.

16. The method of claim 1, further comprising the step of applying electrochemical energy to said implanted medical device.

17. The method of claim 16, wherein said electrochemical energy applied to said implanted medical device comprises a cathodic voltage.
